(19) United States
(12) Patent Application Publication
(10) Pub. No.: US 2009/0076240 A1
(43) Pub. Date: Mar. 19, 2009

(54) COMPOUNDS HAVING ISOCYANATE FUNCTIONAL GROUP SUBSTITUENTS AND COATING COMPOSITIONS COMPRISED THEREOF

(75) Inventors: Jean-Marie Bernard, Saint-Laurent D'Agny (FR); Philippe Barbeau, Genas (FR); Philippe Olier, Lyon (FR)

Correspondence Address: BUCHANAN, INGERSOLL & ROONEY PC, POST OFFICE BOX 1404, ALEXANDRIA, VA, 22313-1404, US

(73) Assignee: Rhodia Operations, Aubervilliers (FR)

(21) Appl. No.: 12/095,239

(22) PCT Filed: Nov. 28, 2006
(86) PCT No.: PCT/EP06/68987

§ 371 Date: Oct. 3, 2008

(30) Foreign Application Priority Data
Nov. 28, 2005 (FR) 0512036

(51) Int. Cl. C08G 18/32; C09K 3/00; C07C 69/74

(52) U.S. Cl. 528/66; 252/182.24; 560/125; 560/126

(57) ABSTRACT

Novel compounds having an average isocyanate functionality of greater than 2 are synthesized by the reaction of a (poly)isocyanate having an average functionality of greater than 2, with a compound X comprising a function B(H)ₙ or B′(H)_{n′} where n is equal to 1 or 2, n′ is equal to 1, 2 or 3, H is a labile hydrogen atom and B is O, S, N, N being a primary or secondary nitrogen atom, —C(═O)—O, —C(═O)—N, or else the groups O═P(O)₂; O═P(O)OR₁; O═P(O)₃; O═P(O)2OR₁; O—P(O)—OR₁, and B′ is —SiR₂R₃R₄, such reaction being carried out with a compound X/[compound X+(poly)isocyanate] weight ratio of at most 50%; compositions comprised thereof are of the hardener type and can be formulated into coatings of the paint or varnish type.

The present invention relates to a compound having isocyanate functionality, to its preparation and to its use in a process for the preparation of a coating, in particular of paint or varnish type, for metal substrates in particular.

The fields of the automobile and aeronautics industries increasingly require very technically advanced coating compositions having improved conditions of use which result in quality finished products.

Compositions of this type include polyurethane formulations comprising two components, one of which is a curing agent of the polyisocyanate type and the other of which is the resin of the polyol type which additionally comprises additives necessary for the coating. These components are formulated in two containers separated for the storing and they are mixed at the time of manufacture of the coating so as to carry out a crosslinking reaction between the polyisocyanate and the polyol.

In order to increase productive output and in particular to accelerate the turnover of vehicles to be painted in paint booths, a demand exists for paint formulations which dry increasingly rapidly. Drying in fact involves two phenomena. There is first of all a chemical phenomenon, which is the formation of a polyurethane network and which is essentially the consequence of the creation of covalent urethane bonds by chemical reaction between the curing agent and the polyol. In addition there is a physical phenomenon, which is reflected by the rate at which the coating cures. This drying, in the physical sense, is related to the specific nature of the constituents used and in particular to the glass transition temperature (Tg) of these.

A recommended and known solution for increasing the rate of drying is to add, to an aliphatic polyisocyanate curing agent generally based on hexamethylene diisocyanate (HDI), a certain amount of cycloaliphatic polyisocyanates in particular based on isophorone diisocyanate (IPDI), such as isophorone diisocyanate trimer (IPDT). However, these IPDI-based polyisocyanate curing agents comprise isocyanate functional groups which are markedly less reactive than those of the polyisocyanates having an aliphatic structure based on HDI. If “physical” drying is accelerated, the chemical crosslinking process is, on the other hand, markedly slowed down.

The presence of unreacted isocyanate functional groups in the coatings thus obtained results in plasticizing of the latter during polishing and finishing operations. The coatings deteriorate, indeed the paint film even detaches, thus creating blemishes which require repairing.

Furthermore, excessively rapid exposure of these coatings to damp conditions results in the appearance of off-white stains due to the reaction of the unreacted isocyanate functional groups with water, which here again makes repairing necessary.

The problem which is thus posed is that of accelerating the drying while retaining the properties of the coating.

The object of the invention is to provide a compound which makes it possible to increase the rate of the drying while retaining good reactivity of the isocyanate functional groups and thus a compound which makes it possible to respond to the problem mentioned above.

With this aim, the invention relates to a compound having isocyanate functionality, exhibiting a mean functionality of greater than 2, resulting from the reaction of a (poly)isocyanate with a mean functionality of greater than 2 with at least one compound X comprising:
either at least one functional group B(H)ₙ in which:
   n is a number equal to 1 or 2,
   H is a labile hydrogen and
   B denotes O, S, N(N being a primary or secondary nitrogen), —C(═O)—O, —C(═O)—N, or the groups

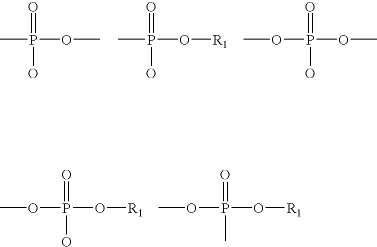

R₁ denoting an alkyl or aralkyl radical which is optionally branched or an alkyl chain interrupted by a heteroatom;
or at least one functional group B′(H)_{n′} in which:
   n′ is a number equal to 1, 2 or 3,
   H is a labile hydrogen and
   B′ denotes —SiR₂R₃R₄, R₂, R₃ and R₄ representing oxygen, an alkyl radical carrying a functional group which reacts with the (poly)isocyanate, or an aralkyl, aryl, —O-alkyl or —O-aralkyl radical, the number of R₂, R₃ and R₄ radicals being such that n′ can indeed comply with the above condition;
   X being in addition a cycloaliphatic, aromatic or heterocyclic compound or a particle of a crosslinked polymer;
   and with the conditions:
that, when B denotes a secondary nitrogen and when X is a cycloaliphatic compound, X is then a compound exhibiting at least two rings;
and that the abovementioned reaction is carried out with a compound X/[compound X+(poly)isocyanate] ratio by weight of at most 50%.

The invention also relates to a composition of the curing agent type which is characterized in that it comprises at least one compound of the above type.

Other characteristics, details and advantages of the invention will become even more fully apparent on reading the description which will follow and also various concrete but nonlimiting examples intended to illustrate it.

It is specified, for the continuation of the description, that, unless otherwise indicated, in the ranges of values which are given, the values at the limits are included.

The compound of the invention comprises isocyanate functionality, that is to say that it exhibits isocyanate functional groups in its structure, and it can in addition have functional groups of urethane, allophanate, urea, biuret, ester or carbonate type.

In addition, this compound exhibits a mean functionality of greater than 2, more particularly of at least 2.5 and more particularly still of at least 2.8. According to preferred embodiments, this mean functionality can be at least 3, more particularly at least 3.2 and more preferably still at least 3.5. This mean functionality is generally at most 20, more particularly at most 15 and more particularly still at most 10.

For this compound, here and for the remainder of the description, the term “mean functionality”, for the isocyanate functional group, is understood to mean the number r:

r=Mn×[I]

in which Mn is the number-average molar mass of the compound having isocyanate functionality and [I] is the concentration of isocyanate functional group, expressed in mol/g.

The mass Mn is determined by gel permeation chromatography.

The compound of the invention is the product of the reaction of at least one abovementioned compound X with a (poly)isocyanate. It should thus be understood that the invention applies to the case where the (poly)isocyanate is reacted with a mixture of several compounds X.

As indicated above, the compound X must exhibit several characteristics.

It must first of all comprise at least one functional group B(H)ₙ or B′(H)_{n′} in which H is a labile hydrogen and B denotes different atoms or groups of atoms.

Thus, B can denote O, the functional group BH being OH in this case; S, case corresponding to the functional group SH; N, nitrogen being primary or secondary, that is to say that the nitrogen atom carries two hydrogen atoms or one hydrogen atom respectively, which corresponds to the functional groups NH₂ or NH respectively, it being possible for B also to be the C(═O)—N group, which corresponds, for BH, to the functional group C(═O)—NH or C(═O)—NH₂ or also C(═O)—NHR′, R′ being an optionally branched alkyl chain or an aryl chain of 1 to 10 carbon atoms generally. The values of B which are mentioned here correspond to preferred embodiments of the invention.

Furthermore, as indicated above, B can denote a group comprising phosphorus, in which case the compound X is, for example, a compound of formula

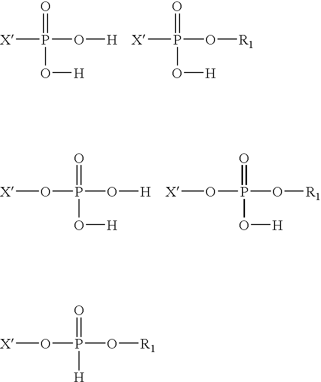

in which X′ is the residue of the compound X and R₁ is as defined above, it being specified here that the alkyl or aralkyl radical comprises more particularly at most 20 carbon atoms, in particular from 1 to 10 carbon atoms. In the case where R₁ denotes an alkyl chain interrupted by a heteroatom, this chain comprises a carbon number which is generally at most 60, more particularly at most 40 and more particularly still at most 35. In this same case, the heteroatom can be more particularly oxygen and the number of heteroatoms is preferably between 1 and 20. The group B as defined in the present paragraph is advantageous in the case of the use of the compounds of the invention in aqueous-phase compositions.

Finally, it should be noted that the invention applies, of course, to the cases where X comprises several functional groups B(H)ₙ or B′(H)_{n′}, it being possible for these functional groups to be identical or different.

This compound X exhibits more particularly a mean functionality equal to or greater than 1 which can, for example, be between 1 and 10 and more particularly 1 and 5 and advantageously between 1 and 3.

This mean functionality of the compound X is given by the number r′:

r′=M/n×[B(H)ₙ] or r′=M′n×[B′(H)_{n′}]

in which M′n is the number-average molar mass of the compound X and [B (H)ₙ] and [B′(H)_{n′}] represent the respective concentrations of functional group B(H)ₙ and B′(H)_{n′}, expressed in mol/g. The mass M′n is determined by gel permeation chromatography. The concentration of functional group B(H)ₙ or B′(H)_{n′} is calculated by a direct potentiometry method in the case of the functional groups in which B is N, —C(═O)—O, —C(═O)—N and the abovementioned phosphorus-comprising groups or an indirect method for the other functional groups, this indirect method consisting in reacting the functional group B(H)ₙ or B′(H)_{n′} with acetic anhydride and in then backtitrating the acetic acid released. An analytical method, such as NMR, can also be used to determine this concentration.

The compound X is furthermore an organic compound having a cycloaliphatic, aromatic or heterocyclic structure. The rings constituting the structure of the compound X can be fused or bonded to one another via aliphatic chains or also via a simple bond. This is the case, as indicated above, when B denotes a secondary nitrogen. In the case of aliphatic chains, the latter are preferably short and optionally branched, for example chains of at most 15 carbon atoms, in particular of at most 10 carbon atoms, more particularly of at most 6 carbon atoms and more particularly still of at most 4 carbon atoms. These rings can comprise short alkyl chains which are optionally branched, for examples chains of at most 10 carbon atoms, in particular of at most 6 carbon atoms, more particularly of at most 4 carbon atoms and more particularly still of at most 2 carbon atoms. The rings can also be bicyclic.

According to a preferred embodiment of the invention, the compound X comprises at least two rings, more particularly at least three. In the case of the use of a mixture of compounds X, it is preferable for at least one of these compounds to be a compound having at least two rings.

Finally, it can be a compound in the form of particles based on a crosslinked polymer.

For the choice of the compound X, compounds having a rigid structure will preferably be selected. This term is understood to mean structures which are sterically hindered or structures having reduced conformational mobility or which are capable of developing strong inter- or intramolecular bonds which may even result in high degrees of crystallinity, and also crosslinked structures, or, finally, compounds having high melting points or high Tg values, for example a Tg of at least 0° C., more particularly of at least 20° C. and more particularly still of at least 40° C.

In order to have a rigid structure within the meaning given above, use can very particularly be made of the compounds X in which at least one and preferably all of the functional groups B(H)ₙ or B′(H)_{n′} are bonded directly to a carbon of the ring of the compound X. These products, after reaction with the (poly)isocyanate, give structures having reduced conformational mobility. According to a specific embodiment, when X exhibits only a single ring, the functional group B(H)ₙ or B′(H)_{n′} [cf. E8/37] is bonded directly to a carbon of this ring.

According to a preferred embodiment of the invention, the functional group B(H)ₙ is the OH functional group. In this case, preference is given to the compounds having a secondary OH functional group or having a primary but hindered OH functional group. The term “hindered” is understood to mean a functional group, the carbon of which in the β-position with regard to the OH functional group is branched and carries at least one alkyl group with at least one carbon atom. Mention may be made, as example of such a case, of the neopentyl or isobutyl structures.

The compound X can thus be chosen from diols.

Examples of compounds of the diol type which are particularly suitable in the implementation of the invention will be given below.

Thus, the compound X can be chosen from optionally substituted bisphenols, in particular A and F, and their hydrogenated derivatives. Mention may in particular be made of hydrogenated bisphenol A.

Mention may also be made of polyphenolic derivatives having an ether bridge of bisphenols. This term is understood to mean the products of formula (1) or (1′):

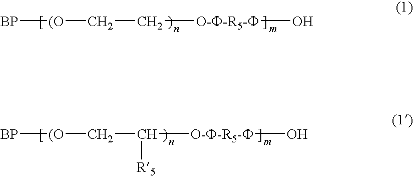

in which BP denotes the residue of a bisphenol radical, φ a benzene nucleus, R₅ a linear or branched alkyl radical, R′₅ a C₁-C₅ alkyl radical, such as methyl, and n and m integers, it being understood that n should preferably have a low value in order to retain a rigid structure in the product, for example of at most 5, preferably of at most 3, and it being possible for m to be, for example, between 1 and 10.

The hydrogenated derivatives of these polyphenolic products can also be used.

Mention may also be made, as compounds X having an OH functional group, of derivatives of cyclopentadiene, dicyclopentadiene and tricyclopentadiene which can be obtained by a hydroformylation reaction of the latter, for example of dicyclopentadiene, followed by a hydrogenation in order to obtain the corresponding polycyclic alcohol, or also derivatives of terpenes, such as terpenylcyclohexanol, and derivatives of the series of the terpenes, such as isobornyl, isocamphyl and isofenchyl. Mention may be made, as example of these norbornadiene derivatives, of 5-ethylidene-2-norbornene or limonene.

Use may be made of cycloalkanes having an OH functional group, more particularly cyclohexanediol, tricyclodecanedimethanol, tricyclodecanediol, tricyclohexylmethanol or also derivatives of terpenylcyclohexanol or of isobornylcyclohexanol, derivatives of adamantane for fused tricyclic compounds or decalindiol in fused bicyclic compounds.

In addition, the reaction products of carboxylic acids with compounds having a blocked hydroxyl functional group are also suitable in the context of the invention as compounds X. The term “compounds having a blocked hydroxyl functional group” is understood to mean compounds having epoxy or carbonate functional groups, in the latter case preferably cyclic carbonate functional groups, such as glycerol carbonate, or also compounds having a dioxolane functional group. Mention may be made, for the latter functional group, as compound, of 2,2-dimethyl-1,3-dioxolane-4-methanol. The reaction concerned here can be either an esterification between the blocked hydroxyl functional groups and the carboxyl functional groups or a reaction by opening of the blocked hydroxyl functional group with release of the hydroxyl functional group. For the latter case, mention may more particularly be made of the reaction of carboxylic acids with compounds having an epoxy functional group.

The carboxylic acids can in particular be acids of formula R₄COOH in which R₄ is an aliphatic, cyclic, polycyclic, aromatic or heterocyclic radical which is optionally branched or substituted.

The compounds having an epoxy functional group can be aliphatic, cycloaliphatic or heterocyclic and can comprise at least one functional group derived from carboxyl functional groups (ester or amide functional groups). These compounds can optionally carry substituents which are optionally branched alkyl chains. Preference is given to the epoxy compounds having at least one ring.

Mention may be made, as examples, for the carboxylic acids, of acetic acid, propionic acid, isobutyric acid, 2,2,2-trimethylacetic (pivalic) acid, benzoic acid, cyclohexanoic acid, terephthalic acid, phthalic acid or cyclohexanedicarboxylic acid.

Mention may be made, still as examples, for the epoxy compounds, of styrene oxide, epichlorohydrin and its derivatives, cyclohexene oxide, exo-2,3-epoxynorbornane (bicyclic structure), 3,4-epoxycyclohexylmethyl 3,4-epoxycyclohexanecarboxylate, methyl 3,4-epoxycyclohexanecarboxylate or 1,3-dimethylpropane-1,3-diol bis(3,4-epoxycyclohexanecarboxylate).

Use may also be made, as compound X having an OH functional group, of the product of the reaction of a compound having epoxy functional group(s) with a phosphate of formula (2) (O)P(OR₆)(OR₇)(OR₈) in which R₆, R₇ and R₈ are identical or different and denote hydrogen, a linear or branched alkyl radical, in particular comprising from 1 to 25 carbon atoms, a cycloaliphatic radical, an aromatic radical, an aralkyl radical or a polyoxyalkylene chain, the number of linear or branched oxyalkylene units of which is between 1 and 25 and the number of carbons of the alkylene chain of which is between 2 and 6, it being possible for this polyoxyalkylene chain preferably to be substituted on its end functional groups by a linear or branched, preferably C₁-C₂₀, alkyl chain or by an aralkyl chain which is optionally branched, the number of carbons of which can be between 7 and 20; and with the condition, in the formula (2), that at least one of R₆, R₇ and R₈ is a hydrogen atom. Preference is given, for the compounds of formula (2), to those comprising a ring.

Use may also be made, as compound X, of the product of the reaction of a compound having an epoxy functional group with a polyaminoether of formula (3)

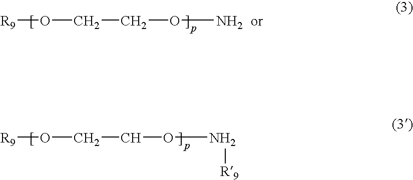

or also of formula (4) R₉—[—O—CH₂—CH₂—O—]_{q}—CH(CH₃)—CH₂—NH₂, R₉ being hydrogen, an alkyl radical, in particular a C₁-C₄ alkyl radical, or a CH₂CH₂NH₂ or CH₂CH(CH₃)NH₂ radical, R′₉ being an alkyl radical, in particular a C₁-C₄ alkyl radical, and p and q being integers between 2 and 10, preferably between 2 and 5; or the product of the reaction of a compound having an epoxy functional group with morpholine or a derivative of the latter.

As indicated above, the compound of the invention results from the reaction of a compound X as described above with a (poly)isocyanate with a mean functionality of greater than 2.

The term “(poly)isocyanate with a mean functionality of greater than 2” is understood to mean compounds which exhibit at least one isocyanurate ring, one biuret unit, one allophanate functional group or one acylurea functional group. It is also understood to mean compounds which exhibit at least one uretidinedione ring and isomers of isocyanurates, such as iminooxadiazinedione. These compounds can be obtained by homo- or heterocondensation of monomers chosen from aliphatic, cycloaliphatic, arylaliphatic, aromatic and heterocyclic isocyanate monomers and in particular, among these, diisocyanates and triisocyanates.

Mention may be made, as nonlimiting examples of aliphatic and cycloaliphatic isocyanates, of the following products:
1,6-hexamethylene diisocyanate (HDI),
1,12-dodecane diisocyanate,
1,3-cyclobutane diisocyanate,
1,3- and/or 1,4-cyclohexane diisocyanate,
1-isocyanato-3,3,5-trimethyl-5-(isocyanatomethyl)-cyclohexane (isophorone diisocyanate) (IPDI),
isocyanatomethyl diisocyanates, in particular 4-isocyanatomethyl-1,8 diisocyanate (TTI),
2,4- and/or 2,6-hexahydrotoluylene diisocyanate (H₆TDI),
hexahydro-1,3- and/or -1,4-phenylene diisocyanate,
perhydro-2,4′- and/or -4,4′-diphenylmethane diisocyanate (H₁₂MDI),
   and generally the hydrogenation products of the aromatic precursors, amines or carbamates,
bis(isocyanatomethyl)cyclohexanes (in particular 1,3- and 1,4-) (BIC),
bis(isocyanatomethyl)norbornanes (NBDI),
2-methyl-1,5-pentamethylene diisocyanate (MPDI),
tetramethylxylylene diisocyanate (TMXDI),
esters of lysine di- or triisocyanate (LDI or LTI),
triisocyanates, such as 4-isocyanatomethyl-1,8-octamethylene diisocyanate.

Mention may be made, as nonlimiting examples of aromatic isocyanates, of:
2,4- and/or 2,6-toluylene diisocyanate (TDI),
2,4′ and/or 4,4′-diphenylmethane diisocyanate (MDI),
1,3- and/or 1,4-phenylene diisocyanate,
4,4′,4″-triphenylmethane triisocyanate, and
oligomers of MDI or of TDI.

However, it is preferable to use the aromatic derivatives only in limited amounts as they can result in coatings which can undergo coloring during aging, in particular in the event of exposure to ultraviolet radiation, for example solar ultraviolet radiation. For this reason, aliphatic and cycloaliphatic derivatives, at least one of the NCO and preferably both of the NCO functional groups of which is/are not directly grafted to the aliphatic ring, are generally preferred.

As will be seen later, it is also possible to use (poly)isocyanates as defined above which are in addition rendered hydrophilic by grafting a suitable hydrophilizing additive.

Use is more particularly made, in the context of the present invention, of (poly)isocyanates exhibiting a mean functionality of at least 2.5 and more preferably still of at least 3. Generally, the mean functionality is at most 30, more particularly at most 15 and more particularly still at most 10; for example, it can be between 3 and 6.

The (poly)isocyanates can be used as a mixture with the abovementioned monomers but, in this case, the content of monomer is at most 50%, in particular at most 30%, more particularly at most 20% and more particularly still at most 10%.

It is also preferable to use (poly)isocyanates of low viscosity, that is to say with a viscosity of at most 40 000 mPa·s, more particularly of at most 20 000 mPa·s, more particularly still of at most 10 000 mPa·s, preferably of at most 5000 mPa·s, and more preferably still of at most 2500 mPa·s. This viscosity corresponds to a measurement performed at a solids content of 100% at 25° C.

The compounds having isocyanate functionality of the invention are obtained by reacting a compound X with a (poly)isocyanate of the type of those described above.

This reaction is carried out with a compound X/[compound X+(poly)isocyanate] ratio by weight of at most 50%. This ratio can in particular be at most 40%, more particularly at most 25% and more particularly still at most 20%. Generally, it is at least 1%, more particularly at least 2% and more particularly still at least 5%.

The respective values of the functionalities of the compound X and of the (poly)isocyanate are chosen in such a way that, on conclusion of the reaction, a compound is obtained which has to be able to be formulatable, that is to say usable under the conditions of the application desired. The term “formulatable” is understood to mean a product which generally exhibits, at a solids content of 70% and at 25° C., a viscosity of at most 20 000 mPa·s, more particularly of at most 10 000 mPa·s and preferably of at most 6000 mPa·s. It should be clearly understood that this value is given here only by way of example and that it cannot be regarded as limiting.

The conditions under which the reaction between the compound X and (poly)isocyanate can be carried out will now be described in more detail.

Generally, the process is carried out in such a way that the B(H)ₙ/NCO molar ratio is between 1 and 50%, preferably between 2 and 30% and advantageously between 3 and 25%. These ratios are chosen by a person skilled in the art according to the molecular weight, the functionality of each of the products involved and the compounds which it is desired to obtain.

Also generally, the synthesis of the compounds of the invention is carried out according to a conventional reaction of isocyanate functional groups with functional groups B(H)ₙ at a temperature of between 20 and 200° C., preferably at a temperature of between 25 and 150° C. In some cases, a catalyst can be added; generally, these catalysts are organometallic compounds having Lewis acid activity. Mention may thus be made of tin derivatives, such as dibutyltin dilaurate, dibutyltin diacetate, zirconium or aluminum acetylacetones, or bismuth acylates (acetate, octoate, and the like), this list of catalysts not being limiting.

The amount of catalyst of use in obtaining the compounds of the invention can be between 0 and 1000 ppm, advantageously between 0 and 500 ppm and more preferably still between 0 and 250 ppm, with respect to the amount of (poly)isocyanates.

The syntheses are carried out in bulk or in solvent, depending on the viscosity of the final compound obtained. The solvent for the synthesis is generally chosen from esters, such as n-butyl acetate, tert-butyl acetate, aromatic solvents, such as Solvesso 100, or ketones, such as methyl isobutyl ketone.

It should be noted that it is possible to distill the compound X and also the abovementioned solvent for the synthesis in order to remove residual traces of water from the reaction medium.

After the general points given above, the synthesis of the compounds will now be described in more detail according to the type of compound having isocyanate functionality which it is desired to prepare. In the description which follows, the level of isocyanate functional groups consumed is expressed as % and corresponds to the following formula:

(L NCO start−L NCO finish/L NCO start)×100

with L NCO start representing the value of the assay of isocyanate functional groups in the starting reaction mixture and L NCO finish representing the value of the assay of isocyanate functional groups in the reaction mixture at the time of measurement.

The level of isocyanate functional groups consumed is measured according to the standard NF T 52-132, which is a method by quantitative determination with N,N-dibutylamine (DBA).

From this quantitative determination, it is also possible to estimate the degree of conversion of starting isocyanate compound. When an isocyanate functional group of this isocyanate compound has been converted during the reaction, then the molecule of this isocyanate is also consumed. Thus, for the hexamethylene diisocyanate monomer, which is a diisocyanate, the degree of conversion of isocyanate corresponds to approximately double the value found for the assay of isocyanate functional groups.

In the case of a compound according to the invention having an isocyanate functional group hut also having a urethane functional group, the preparation process comprises the following stages:
(a) introduction of the or of the mixture of (poly)isocyanate(s) into a reactor equipped with a stirring system and monitoring devices;
(b) optional addition of solvent;
(c) addition to the (poly)isocyanate or to the mixture of (poly)isocyanates of the compound X or of the mixture of compounds X having B(H)ₙ functional groups, B(H)ₙ being, in the present case (preparation of a compound having isocyanate/urethane functional groups), an OH functional group;
(d) placing the reaction mixture under stream of an inert gas, such as nitrogen or argon;
(e) optional addition to the mixture of reaction additives, such as antioxidants or stabilizing agents of trialkyl or triaryl phosphite type or hindered phenolic aromatic compounds, such as 2,6-di(tert-butyl)phenol, or oligomeric derivatives of these compounds;
(f) reaction at a temperature of between 80 and 130° C. until the degree of conversion of isocyanate functional groups is equal to the molar amount of functional groups B(H)ₙ to cause to react. Thus, in the present case (formation of urethane bonds), the amount of isocyanate functional groups consumed will be equal to at least 90% of the molar amount of functional groups B(H)ₙ, preferably greater than 95% of this amount and more preferably still equal to the total amount of functional groups B(H)ₙ introduced;
(g) recovery of the product once the degree of conversion is achieved.

In the case of a compound according to the invention having isocyanate functional group(s) but also having allophanate functional group(s), the preparation process here again employs a compound X (or a mixture of compounds X) having an OH functional group.

The process comprises the stages (a) to (e) described above and also a stage of addition, after stage (d), of a Lewis acid catalyst (dibutyltin dilaurate or zirconium acetylacetonate) in the amounts indicated above.

The process additionally comprises a reaction stage (f) which is carried out here at a temperature of between 100 and 150° C. until the degree of conversion of isocyanate functional groups is equal to approximately twice the molar amount of functional groups B(H)ₙ to cause to react. This is because a compound having an allophanate functional group is the reaction product of a compound having an isocyanate functional group with a compound having a urethane functional group, the latter being itself the product of the reaction of a compound having an isocyanate functional group with a compound having a hydroxyl functional group. In order to obtain the conversion to allophanate, two moles of isocyanate functional group are thus theoretically consumed per one mole of OH functional group and, in practice here, the reaction is carried out so as to consume an amount of isocyanate functional groups equal to at least one mole and between one and two, this being the case according to the properties which it is desired to obtain for the compound X.

The synthesis of compounds having isocyanate functionality additionally comprising urea functional groups involves compounds X for which B(H)ₙ is either a primary amine or a secondary amine. The preparation process takes place in the same way as for the syntheses of the compounds having isocyanate and urethane functional groups, except that the reaction temperature is between ambient temperature and 80° C. The reaction does not require specific catalysts. The reaction is halted when the degree of conversion of isocyanate functional groups is equal to the molar amount of functional groups B(H)ₙ (in the present case, primary or secondary amine) to cause to react. Thus, in the present case of formation of urea bonds, the amount of isocyanate functional groups consumed will be equal to at least 90% of the molar amount of functional groups B(H)ₙ or B′(H)_{n′}, preferably equal to at least 95% of this amount and more preferably still equal to the total amount of functional groups B(H)ₙ introduced.

The synthesis of polyisocyanate compounds having isocyanate functionality additionally comprising biuret functional groups involves compounds X for which B(H)ₙ is either a primary amine or a secondary amine. The synthesis is carried out in the same way as for the syntheses of the compounds having isocyanate and urethane functional groups, except that the reaction temperature is between 100 and 150° C. The reaction can be catalyzed with the addition of acid compounds, such as propionic acid or dialkyl phosphates. A biuret compound is the reaction product of an isocyanate compound with a compound having a urea functional group and the latter is itself the reaction product of a compound having an isocyanate functional group with an amine. In order to obtain the conversion to biuret, two moles of isocyanate functional group are thus theoretically consumed per one mole of amine functional group and, in practice here, the reaction is carried out so as to consume an amount of isocyanate functional groups equal to at least one mole and between one and two, this being the case according to the properties which it is desired to obtain for the compound X.

In the case of the compounds X for which the functional groups B(H)ₙ are SH functional groups, use will then be made of the same type of process as that described in order to obtain the compounds having isocyanate and urethane functional groups. The consumption of the isocyanate functional groups will be the same as for that of the urethane functional groups, if the reaction is halted at the stage of the thiourethane compounds. It will correspond to the degree of conversion of the NCO functional groups of the allophanate process if it is desired to obtain thioallophanate compounds.

As regards the compounds X for which the functional groups B(H)ₙ are amide functional groups, the process will be the same, except that the operating conditions will be a reaction temperature of between 100 and 150° C. and the optional presence of a Lewis acid catalyst. The reaction is halted when the degree of conversion of isocyanate functional groups is equal to the molar amount of functional groups B(H)ₙ (in this case, primary or secondary amide) which it is desired to cause to react. The amide functional groups can result from a reaction between isocyanate functional groups and acid functional groups.

In the case of compounds X having different functional groups B(H)ₙ or B′(H)_{n′}, the operating conditions will be adjusted to the compound which it is desired to obtain. Generally, it should be noted that the conditions for reactions of a compound having an isocyanate functional group with a compound having a labile hydrogen of the compound X type are well known to a person skilled in the art and reference may be made, on this subject, to the general work “Methoden der Organischen Chemie”, Houben Weyl, Georg Thieme Verlag 1983.

In all the cases which have just been described, the structures of the compounds are confirmed by known analytical techniques, such as infrared spectroscopy or proton or carbon nuclear magnetic resonance (NMR) spectrometry.

The invention also relates to a composition or formulation of the curing agent type which is characterized in that it comprises a compound having isocyanate functionality of the above type or a mixture of these compounds. This compound can be the sole component of the composition of the curing agent type; nevertheless, this composition can additionally comprise a (poly)isocyanate or a (poly)isocyanate mixture within the meaning given above, which implies that everything which was described above on the subject of (poly)isocyanates, both for their nature and for their viscosity in particular, also applies here. This additional (poly)isocyanate or (poly)isocyanate mixture can be different from that used for the preparation of the compound having isocyanate functionality of the invention. The addition of this additional (poly)isocyanate or (poly)isocyanate mixture makes it possible to vary in particular the viscosity of the composition and/or its assay of isocyanate functional group. Preferably, the content of compound having isocyanate functionality in the composition of the curing agent type is between 100% and 5%, more particularly between 100% and 10% and more particularly still between 100% and 25%, this content being expressed as weight of compound having isocyanate functionality with respect to the total weight of the composition of the curing agent type.

The composition of the curing agent type of the invention can be used for the preparation of coatings of paint or varnish type in the solvent phase or in the aqueous phase.

In the case of a use in the solvent phase, very particularly in the case where stability on storage of several days to several months is desired, it is preferable to use an unreactive solvent. For example, this can be a solvent of the following types: ester (butyl acetate), ketone, acylated or dialkylated glycol ether or substituted aromatic solvents, such as xylene. They are solvents which are common in the formulation of isocyanates, preferably having a low amount of water.

In the case of use in the aqueous phase, the composition can comprise an additive which makes it possible to emulsify it or to render it dispersible or water-soluble. Several embodiments and alternative forms can then be envisaged.

In the case of a first embodiment, the composition of the curing agent type comprises a hydrophilic additive of the unreactive type, that is to say that this additive is present as a mixture with the composition without the reaction having occurred between this additive and the compound having isocyanate functionality of the composition. Mention may be made, as additive of this type, of those described in the documents WO 97/31960 and FR 2855768-A1, to the teaching of which reference may be made. These additives exhibit an anionic functional group and advantageously a polyethylene glycol chain fragment of at least one, preferably of at least 5, ethylene oxide units.

Mention may more particularly be made, among these additives, of those below:

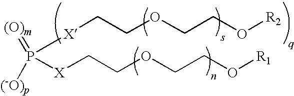

with, when q is equal to zero:

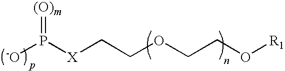

and where p represents zero or an integer between 1 and 2 (closed intervals, that is to say comprising the limits);
where m represents zero or an integer between 1 and 2 (closed intervals, that is to say comprising the limits);
where the sum p+m+q is at most equal to three;
where the sum 1+p+2 m+q is equal to three or to five;
where X and X′, which are identical or different, represent an arm comprising at most two carbon links;
where n and s, which are identical or different, represent an integer chosen between 5 and 30, advantageously between 5 and 25, preferably between 9 and 20 (closed intervals, that is to say comprising the limits);
where R₁ and R₂, which are identical or different, represent a hydrocarbon radical advantageously chosen from aryls and alkyls which are optionally substituted, in particular by halogen atoms, in particular fluorine.

In the case of a second embodiment, the composition of the curing agent type comprises a hydrophilic additive of the reactive type, that is to say that this additive is present in the composition but while being grafted to the compound having isocyanate functionality of the composition. In the case of this second embodiment, several alternative forms can then be envisaged.

According to a first alternative form, the choice is made of a specific compound X, that is to say a hydrophilic compound X, which is reacted with a (poly)isocyanate in the manner described above. Use may be made, as compounds X of this type, of those having an OH functional group resulting from the reaction of a compound having epoxy functional group(s) with a phosphate of formula (2) and those resulting from the reaction of a compound having an epoxy functional group with a polyaminoether of formula (3) or of formula (4) which are described above. In the latter case, a composition is obtained which is either self-emulsifiable or, if it is not self-emulsifiable to a sufficient degree, which can be rendered self-emulsifiable by further addition to the composition of an unreactive hydrophilic additive of the type which was described above.

According to another alternative form of this second embodiment, the grafted additive can be introduced by the (poly)isocyanate itself. Mention may be made, as additives which can be grafted to (poly)isocyanates, of the hydrophilic additives mentioned in patent U.S. Pat. No. 4,663,377, to the teaching of which reference may be made. The (poly)isocyanate thus treated can be reacted with a hydrophobic compound X and, in this case, a composition is here again obtained which is either self-emulsifiable or, if it is not self-emulsifiable to a sufficient degree, which can be rendered self-emulsifiable by addition of a hydrophilic additive of the unreactive type as described above.

Finally, it is also possible to react a (poly)isocyanate grafted with the hydrophilic additive with a compound X which is itself hydrophilic, in order likewise to obtain a self-emulsifiable composition or a composition for which the self-emulsifying property can also be improved in the manner described above.

The invention also covers a process for the preparation of a coating on a substrate which is characterized in that a composition of the curing agent type as described above is mixed with at least one compound carrying at least one functional group having a mobile hydrogen chosen from hydroxyl, primary amine and secondary amine functional groups and the SH functional group or with a compound comprising precursor functional groups capable of releasing hydroxyl functional groups, and the mixture obtained is applied to the substrate. Crosslinking subsequently takes place.

This is in fact a process for the preparation of a polyurethane-based coating by reaction of a curing agent with a resin, a process well known to a person skilled in the art.

It should be noted that the crosslinking of the curing compound of the invention can also take place by itself by the action of atmospheric humidity.

The abovementioned mixing of the composition of the curing agent type and of the compound carrying at least one functional group having a mobile hydrogen takes place in the presence of a solvent and also generally and in a way known to a person skilled in the art in the presence of organic or inorganic additives, such as a rheology additive, a solvent, a thickening agent, surfactants or catalysts, according to the properties desired.

It is also possible, during the formation of the mixture, to add other compounds, such as aminoplast or epoxy resins.

Preferably, the compounds are chosen from polyols, which can be used alone or as a mixture. These can advantageously be acrylic, polyester or polyurethane polymers or blends of these polymers. Mention may also be made of polyethers, which are not preferred, however.

Mention may be made, as precursor functional groups capable of releasing hydroxyl functional groups, of epoxy, carbonate or dioxolane functional groups. These precursor functional groups release the hydroxyl functional groups by reaction with an appropriate nucleophile, such as an amine or water, optionally in the presence of a catalyst, which can be an acid compound or a Lewis acid, in an amount by weight which can be, for example, between 50 and 5000 ppm, more particularly between 100 and 500 ppm, an amount expressed as weight of catalyst with respect to the solids content of the composition of the curing agent type and of the compound carrying at least one functional group having a mobile hydrogen.

Once the abovementioned mixture has been deposited, the reaction between the composition of the curing agent type and the compound carrying at least one functional group having a mobile hydrogen can take place at ambient temperature or, more frequently, under hot conditions at a temperature which can be between 30° C. and 300° C., preferably between 40° C. and 250° C. and more preferably still between 50° C. and 150° C. The temperature and the crosslinking time are adjusted according to the substrate. In the case of temperature-sensitive substrates, use will more particularly be made of crosslinking catalysts.

The substrate can be a metal substrate, for example made of aluminum or steel, in particular of stainless steel. This can also be a substrate made of plastic polymer. The substrate can be a motor vehicle bodywork component, for example. The substrate can also be a substrate made of wood or of paper. Furthermore, the substrate can already comprise a protective layer of the paint or varnish type.

The compound of the invention has drying acceleration properties. For this reason, the invention also covers the use of this compound as drying accelerator in a process for the preparation of a coating on a substrate, in which process a composition of curing agent type comprising said compound is mixed with a compound carrying at least one functional group having a mobile hydrogen chosen from hydroxyl, primary amine and secondary amine functional groups and the SH functional group, and the mixture thus obtained is deposited on the substrate.

That which was said above on the subject of the compound carrying at least one functional group having a mobile hydrogen, of the process for the preparation of the coating and of the substrate applies here, of course.

In addition, it should be noted that the compound of the invention, in addition to the drying acceleration property, makes it possible to more rapidly obtain other properties, such as the hardness and the chemical resistance. It can thus offer a good compromise of properties; in particular, it can also exhibit an improved pot life.

Examples will now be given.

The products used in these examples are as follows:
Tolonate HDT LV2: isocyanurate-based HDI-based polyisocyanate of low viscosity sold by Rhodia, with a content of isocyanate functional group of the order of 22.5% and with a viscosity of approximately 600+/−150 mPa·s at 25° C., functionality of isocyanate functional group of the order of 3.3
Tolonate HDT: isocyanurate-based HDI-based polyisocyanate sold by Rhodia, with a content of isocyanate functional group of the order of 22% and with a viscosity of 2400+/−400 mPa·s at 25° C., functionality of isocyanate functional group of the order of 3.6
Tolonate XFD 90 B: isocyanurate-based HDI-based polyisocyanate sold by Rhodia, with a content of isocyanate functional group of the order of 17.4% approximately and with a viscosity of 2000+/−1000 mPa·s at 25° C.
Tolonate HDB: biuret-based HDI-based polyisocyanate sold by Rhodia, with a content of isocyanate functional group of 22% by weight, with a viscosity of 9000+/−2000 mPa·s at 25° C. and with a functionality of 3.7
Tolonate HDB LV: biuret-based HDI-based polyisocyanate of low viscosity sold by Rhodia, with a content of isocyanate functional group of the order of 23.5%, with a viscosity of 2000+/−500 mPa·s at 25° C. and with a functionality of isocyanate functional group of the order of 3.5
Hydrogenated bisphenol A: 2,2-bis(4-hydroxycyclohexyl)propane (HBPA), also known as 4,4′-isopropylidenedicyclohexanol (CAS RN: 80-04-6), molecular weight 240 g, solid product supplied by Maruzen, composed of two rings connected to one another via a branched aliphatic chain comprising 3 carbons and having two secondary hydroxyl functional groups. The OH functionality is 2.
Tricyclodecanedimethanol (TCDM): viscous liquid mixture of isomers sold by Celanese, the compounds of which are composed of 3 rings placed side by side and of two primary hydroxyl functional groups. The OH functionality is 2.
Rhodiantal: isobornylcyclohexanol (IBCH): viscous liquid mixture of isomers sold by Rhodia, the compounds of which are composed of 3 rings, including 2, placed side by side, carrying aliphatic chains having 1 carbon, and 1 ring connected to the other two via a simple bond, this ring carrying a single secondary hydroxyl functional group. The average molecular weight is 236.4 g. The hydroxyl number is between 215 and 235 mg KOH/g.
n-Butyl acetate: n-BuOAc
Joncryl SCX 922: acrylic polyol with a solids content of 80% and with an OH content of 4.2%
Synocure 852 BA 80: acrylic polyol with a solids content of 80% and with an OH content of 4.1%
DBDL: dibutyltin dilaurate

### EXAMPLE 1

This example relates to the preparation of a compound according to the invention having isocyanate functionality and additionally comprising urethane functional groups.

450.5 g of Tolonate HDT LV2, 48.5 g of HBPA (0.203 mol) and 111.8 g of n-butyl acetate are successively charged to a reactor under a nitrogen stream. The NCO assay of the starting Tolonate HDT LV2 is 0.537 mol per 100 g. The HBPA/(HBPA+starting isocyanate) ratio is 9.72%.

The mixture is heated at 120° C. for 3 hours. The NCO assay changes from 0.537 mol per 100 g for the starting Tolonate HDT LV2 to 0.331 for the reaction mixture at the end of the reaction.

610.8 g of formulation of compound according to the invention with a solids content of 82% in n-butyl acetate are recovered.

The NCO assay of the formulation of compound according to the invention is 0.33 mol per 100 g. Its viscosity is 1305 mPa·s at 25° C. for a solids content of 82% in n-butyl acetate.

The product is separated by gel permeation chromatography (GPC) on a polymer gel column using dichloromethane as elution solvent and analyzed by infrared.

The level of residual HDI monomer given by GPC is 0.48%.

The functionality of NCO functional groups is 4.

### EXAMPLES 2 AND 3

These examples relate to the preparation of compounds according to the invention having isocyanate functionality and additionally comprising urethane functional groups.

The preparation is carried out as for example 1 but the operating conditions and the HBPA/(starting polyisocyanate+HBPA) ratio are varied. The results are presented in table 1 below.

TABLE 1

| | | | | | | | Viscosity | |
|---|---|---|---|---|---|---|---|---|
| | | | | | | | at | |
| | A1 | A2 | R | | SC | | 25° C. in | |
| Ex. | (g) | (g) | (%) | T/t | (%) | Assay | mPa≅s | Level |
| 2 | 1042 | 76.8 | 6.9 | 120° C. | 100 | 0.466 | 8135 | 0.2 |
| | | | | 30 min + | | | | |
| | | | | 100° C./2 H | | | | |
| 3 | 432.7 | 47.5 | 9.9 | 130° C. | 85.7 | 0.356 | 2079 | 0.28 |
| | | | | 30 min + | n-BuOAc | | | |
| | | | | 1 H 120° C. + | | | | |
| | | | | 3 H15 | | | | |
| | | | | 100° C. | | | | |

A1: Amount of Tolonate HDT LV2
A2: Amount of HBPA
R: HBPA/(HBPA + HDT LV2) ratio by weight
T: Reaction temperature;
t: reaction time
SC: Solids content
Assay: NCO assay in mol per 100 g
Level: Level of residual HDI monomer

### EXAMPLE 4

This example relates to the preparation of a compound according to the invention having isocyanate functionality and comprising allophanate functional groups.

372.4 g of Tolonate HDT LV2 and 27.4 g of HBPA (0.114 mol) are successively charged to a reactor under a nitrogen stream. 200 ppm of dibutyltin dilaurate with respect to the Tolonate HDT LV2 are added. The NCO assay of the starting Tolonate HDT LV2 is 0.55 mol per 100 g. The HBPA/(HBPA+starting isocyanate) ratio by weight is 6.85%.

The mixture is heated at 110° C. for 48 h. The NCO assay of the reaction mixture at the end of the reaction is 0.377 mol per 100 g.

400 g are recovered.

The NCO assay of the formulation of compound according to the invention having allophanate functional groups is 0.377 mol per 100 g. Its viscosity is 14 000 mPa·s at 25° C. and with a solids content of 90% in n-butyl acetate and 800 mPa·s at 25° C. for a solids content of 70% in n-butyl acetate.

The product is separated by chromatography by GPC on a polymer gel column using dichloromethane as elution solvent and analyzed by infrared.

The level of residual HDI monomer given by GPC is 0.5%.

The functionality of NCO functional groups is 8.

### EXAMPLE 5

This example relates to the preparation of a compound according to the invention having isocyanate functionality and comprising urethane functional groups.

The preparation is carried out as for example 1 but using IBCH instead of HBPA.

459 g of Tolonate HDT LV2 and 46.3 g of IBCH (0.196 mol) are successively charged to a reactor under a nitrogen stream. The NCO assay of the starting Tolonate HDT LV2 is 0.55 mol per 100 g. The IBCH/(IBCH+starting isocyanate) ratio by weight is 9.2%.

The mixture is heated at 110° C. for 7 hours 30. The NCO assay changes from 0.496 mol per 100 g for the starting reaction medium to 0.466 mol per 100 g at the end of the reaction.

482 g of product are recovered.

The NCO assay of the formulation of compound according to the invention having a urethane functional group is 0.466 mol per 100 g. Its viscosity is 2057 mPa·s at 25° C.

The product is separated by chromatography by GPC on a polymer gel column using dichloromethane as elution solvent and analyzed by infrared.

The level of residual HDI monomer given by GPC is 0.2%.

The functionality of NCO functional groups is 3.

### EXAMPLE 6

This example relates to the preparation of a compound according to the invention having isocyanate functionality and comprising urethane functional groups.

The preparation is carried out as for example 5 but using Tolonate HDT instead of Tolonate HDT LV2.

374.4 g of Tolonate HDT (1.95 mol of NCO) and 38.1 g of IBCH (0.16 mol) are successively charged to a reactor under a nitrogen stream. The NCO assay of the starting Tolonate HDT is 0.52 mol per 100 g. The IBCH/(IBCH+starting isocyanate) ratio by weight is 9.2%.

The mixture is heated at 110° C. for 6 hours 15. The NCO assay changes from 0.471 mol per 100 g for the starting reaction medium to 0.435 mol per 100 g at the end of the reaction.

389.7 g of product are recovered.

The NCO assay of the formulation of compound according to the invention having a urethane functional group is 0.435 mol per 100 g. Its viscosity is 8498 mPa·s at 25° C. for a solids content of 100%.

The product is separated by chromatography by GPC on a polymer gel column using dichloromethane as elution solvent and analyzed by infrared.

The level of residual HDI monomer given by GPC is 0.2%.

The functionality of NCO functional groups is 3.5.

### EXAMPLE 7

This example relates to the preparation of a compound according to the invention having isocyanate functionality and comprising urethane functional groups.

The preparation is carried out as for example 1 but using tricyclodecanedimethanol (TCDM) instead of HBPA.

300 g of Tolonate HDT LV2 and 21.6 g of TCDM (0.11 mol) are successively charged to a reactor under a nitrogen stream. The NCO assay of the starting Tolonate HDT LV2 is 0.55 mol per 100 g. The TCDM/(TCDM+starting isocyanate) ratio is 6.7%.

The mixture is heated at 80° C. for 2H. The percentage of isocyanate functional groups consumed is 13.3%.

320 g of product are recovered.

The NCO assay of the formulation of compound according to the invention having urethane functional groups is 0.444 mol per 100 g. Its viscosity is 1220 mPa·s at 25° C.

### EXAMPLE 8

This example relates to the preparation of a compound according to the invention having isocyanate functionality and comprising urethane and biuret functional groups.

271.2 g of n-butyl acetate, 101 g of HBPA, 0.1 g of trimethylolpropane, 0.1 g of 1,6-hexanediol, 0.1 g of phthalic anhydride and 0.1 g of maleic anhydride are successively charged to a three-necked reactor. The stirred reaction medium is heated to a temperature of approximately 130° C. At 125° C., the combined reactants are dissolved. Azeotropic distillation is carried out in order to remove the water present in the reactants and the condensation water of the esterification reaction. After reacting for 1H 30, the temperature of the reaction medium is lowered to 110° C. and then 983.4 g of Tolonate HDB, the measured NCO assay of which is 0.527 mol of NCO functional groups per 100 g, are added. The total number of moles of isocyanate functional groups is thus 5.182 mol and the total number of moles of hydroxyl functional groups is 0.76. The molar ratio of the hydroxyl functional groups with respect to the isocyanate functional groups is 14.66%. The HBPA/HDB ratio by weight is 10.27%.

The NCO assay of the reaction medium is regularly monitored. It is 0.331 mol of NCO per 100 g after reacting for 2H 20, 0.327 after reacting for 2H 20 and 0.326 after reacting for 4H 30.

The reaction is halted and the solids content is adjusted to 80% by weight by addition of n-butyl acetate.

The finished product exhibits the following characteristics:
NCO assay=13.69% by weight
Viscosity=9140 mPa·s at 25° C., measured by the “falling ball” method
The flashpoint of the formulation is 48° C.

The formulation comprises the following predominant isocyanate compounds:
20% by weight of n-butyl acetate
polyisocyanate oligomers based on HDI biuret
   17% by weight of true HDI biuret (3 isocyanate functional groups, 3 hexamethylene chains and one biuret functional group)
   8% by weight of HDI bisbiuret (4 isocyanate functional groups, 5 hexamethylene chains and two biuret functional groups)
   1.5% of true HDI dimer (2 isocyanate functional groups, 2 hexamethylene chains and one uretidinedione functional group)
   0.24% by weight of HDI monomer
polyisocyanate oligomers based on HDI biuret which are heavier than the bisbiuret
and polyisocyanate oligomers based on HDI biuret which have HBPA urethane functional groups.

The last two points represent the remainder to 100% by weight, i.e. 53.26% by weight.

The functionality of isocyanate functional groups is at least equal to 4.5.

### EXAMPLE 9

The preparation is carried out as for example 8, except that the amounts of starting material are as follows:

| n-Butyl acetate | 224.8 g |
|---|---|
| Tolonate HDB | 800.2 g |
| HBPA | 98.5 g |
| Trimethylolpropane | 0.1 g |
| 1,6-Hexanediol | 0.1 g |
| Phthalic anhydride | 0.1 g |
| Maleic anhydride | 0.1 g |
| OH/NCO molar ratio | 18% |
| HBPA/HDB ratio by weight | 12.37% |

The finished product exhibits the following characteristics:
NCO assay=12.39% by weight
Viscosity=18 664 mPa·s at 25° C., measured by the “falling ball” method
The color, expressed in Hazen units, is 60
Solids content 79.6%

The formulation comprises the following predominant isocyanate compounds:
20.4% by weight of n-butyl acetate
polyisocyanate oligomers based on HDI biuret
   14.1% by weight of true HDI biuret (3 isocyanate functional groups, 3 hexamethylene chains and one biuret functional group)
   6.7% by weight of HDI bisbiuret (4 isocyanate functional groups, 5 hexamethylene chains and two biuret functional groups)
   1.2% of true HDI dimer (2 isocyanate functional groups, 2 hexamethylene chains and one uretidinedione functional group)
   0.27% by weight of HDI monomer
polyisocyanate oligomers based on HDI biuret which are heavier than the bisbiuret
and polyisocyanate oligomers based on HDI biuret which have HBPA urethane functional groups.

The last two points represent the remainder to 100% by weight, i.e. 57.33%.

The functionality of isocyanate functional groups is at least equal to 5.

### EXAMPLE 10

The preparation is carried out as for example 8, except that the amounts of starting material are as follows:

| n-Butyl acetate | 400 g |
|---|---|
| Tolonate HDB | 967.2 g |
| HBPA | 149 g |
| Trimethylolpropane | 0.14 g |
| 1,6-Hexanediol | 0.14 g |
| Phthalic anhydride | 0.14 g |
| Maleic anhydride | 0.14 g |
| OH/NCO molar ratio | 22.23% |
| HBPA/HDB ratio by weight | 15.4% |

The finished product exhibits the following characteristics:
NCO assay=10.5% by weight
Viscosity=12 273 mPa·s at 25° C., measured by the “falling ball” method
The color, expressed in Hazen units, is 60
Solids content 73.6%

The functionality of isocyanate functional groups is at least equal to 5.

### EXAMPLE 11

The preparation is carried out as for example 8, except that the amounts of starting material are as follows:

| n-Butyl acetate | 400 g |
|---|---|
| Tolonate HDB | 726.1 g |
| HBPA | 149 g |
| Trimethylolpropane | 0.14 g |
| 1,6-Hexanediol | 0.14 g |
| Phthalic anhydride | 0.14 g |
| Maleic anhydride | 0.14 g |
| OH/NCO molar ratio | 29.6% |
| HBPA/HDB ratio by weight | 20.5% |

The finished product exhibits the following characteristics:
NCO assay=8.19% by weight
Viscosity=46 953 mPa·s at 25° C., measured by the “falling ball” method
The color, expressed in Hazen units, is 60
Solids content 68.6%

The functionality of isocyanate functional groups is at least equal to 5.

### EXAMPLES 12 TO 17

### Compositions Comprising Compounds Having Isocyanate Functionality of the Invention

The compositions are prepared from a compound having isocyanate functionality of the invention and from another polyisocyanate. The solids content of the formulations is 80%. The solvent is n-butyl acetate. The natures and the amounts of the constituents of the composition are shown in the table below:

| | Compound having | |
|---|---|---|
| | isocyanate | |
| | functionality of the | |
| | invention (amount in | Other polyisocyanate |
| Composition | g) | (amount in g) |
| Example 12 | Example 10 (51.32) | Tolonate HDB (16.4) |
| Example 13 | Example 10 (50) | Tolonate HDT (16.1) |
| Example 14 | Example 10 (50.8) | Tolonate HDB LV (16.21) |
| Example 15 | Example 10 (50.25) | Tolonate HDT LV2 (16.08) |
| Example 16 | Example 11 (36.4) | Tolonate HDB (20.86) |
| Example 17 | Example 11 (34.7) | Tolonate HDB LV (19.7) |

The characteristics of the compositions are shown in the table below:

| | NCO assay in NCO | Viscosity in |
|---|---|---|
| Composition | mol/100 g | mPa≅s at 25° C. |
| Example 12 | 0.31 | 11 746 |
| Example 13 | 0.312 | 9397 |
| Example 14 | 0.324 | 8571 |
| Example 15 | 0.323 | 6877 |
| Example 16 | 0.323 | 25 875 |
| Example 17 | 0.327 | 16 550 |

The examples which follow illustrate the use of comparative curing compositions and curing compositions according to the invention in applications of varnish type. These examples refer to tests which are described below.

Pot life: the value of the pot life is obtained by virtue of the measurement of the viscosity with the DIN 4 cup. The value of the pot life corresponds to the time necessary for this viscosity to double.

Drying time: the drying time is determined according to the German method DIN 53150. The times T2 and T3 which were measured according to the standard are shown below. T2 corresponds to the time at the end of which a paper no longer adheres to the surface of the varnish film after having been subjected to the pressure of a weight of 20 g for approximately 1 minute. T3 is determined in the same way as T2 with a weight of 200 g.

Gloss: this measurement is characteristic of the homogeneity and of the appearance of the films. It is performed after drying for 7 days in an air-conditioned room at 20° C. using an Erichsen Model S40 glossmeter.

Persoz hardness: the Persoz hardness measurements are performed in an air-conditioned room at 23±3° C. and a relative humidity of 50±10%. The device used is a type 300 test pendulum from Erichsen with a launching stop and automatic counting. The principle of the hardness pendulum is based on the oscillations of a pendulum placed on the film. The number of oscillations increases as the varnish increases in hardness and dryness. The measurement of gain in hardness is the number of oscillations which the pendulum takes to dampen and then to stop. The duration of an oscillation is one second. The test is finished when the damping of the oscillations reaches an amplitude of 4°. The measurements are carried out regularly during drying, at 1, 3 and 7 days.

Methyl ethyl ketone test: this test characterizes the resistance to solvents, in this instance methyl ethyl ketone. The test is carried out after 7 days.

Impact strength: this test makes it possible to demonstrate the brittle nature of a coating. The test consists in subjecting the coating, after drying for 7 days, to the impact of a striking element with specific dimensions and a specific weight, the drop height of which can be adjusted. The minimum height from which the film of paint or of varnish no longer exhibits cracking or flaking is determined. The value is set at 100 cm for the AFNOR standard and at 80 cm for the ASTM standard.

### EXAMPLES 18 TO 20

These examples relate to the use of a comparative curing composition and curing compositions according to the invention in an application of 2K varnish type in the solvent phase for the repairing of motor vehicles.

The characteristics of the curing compositions, which are composed of a part A based on a polyol and of a part B based on a comparative compound having isocyanate functionality or compounds having isocyanate functionality according to the invention, are given in table 2 below.

In the case of example 18, the comparative compound having isocyanate functionality is Tolonate XFD, which is an aliphatic isocyanate of high functionality having good drying properties. Examples 19 and 20 comprise, as compounds having isocyanate functionality, the compounds of examples 2 and 1 above respectively.

TABLE 2

| | Example 19 | | |
|---|---|---|---|
| | comparative | Example 19 | Example 20 |
| Part A | | | |
| Joncryl SCX 922 | 78.00 | 78.00 | 76.00 |
| DBDL, Fluka | 1 | 1 | 1 |
| (1% in butyl acetate) | | | |
| Butyl acetate | 45 | 41.9 | 44 |
| Part B | | | |
| Tolonate XFD | 41.20 | | |
| Compound of example 2 | | 36.80 | |
| Compound of example 1 | | | 50.00 |

Crosslinking is carried out at 23° C. with 55% relative humidity. The viscosity with the DIN 4 cup is adjusted to 23 s.

The properties of the varnishes obtained, measured according to various tests, are given below.

Pot life: the results of this test are given in the following table 3.

TABLE 3

| | Example 18 | | |
|---|---|---|---|
| | comparative | Example 19 | Example 20 |
| Pot life (min) | 40 | 42 | 40 |

Similar pot lives are thus recorded for the three examples.

### Table 4

Drying time: the results of this test are given in the following table 4.

TABLE 4

| | Example 18 | | |
|---|---|---|---|
| | comparative | Example 19 | Example 20 |
| T2 (min) | 200 | 166 | 133 |
| T3 (min) | 230 | 175 | 171 |

A significant reduction in the drying times T2 and T3 is recorded for the compositions according to the invention.

Gloss: the results of this test are given in the following table 5.

TABLE 5

| Example 18 | | |
|---|---|---|
| comparative | Example 19 | Example 20 |
| 94 | 94 | 94 |

All the compositions exhibit a satisfactory appearance.

Persoz hardness: the results of this test are given in the following table 6.

TABLE 6

| | Example 18 | | |
|---|---|---|---|
| Time | comparative | Example 19 | Example 20 |
| 1 day | 72 | 67 | 102 |
| 3 days | 67 | 81 | 100 |
| 7 days | 102 | 125 | 139 |

Methyl ethyl ketone test: the results of this test are given in the following table 7.

TABLE 7

| Example 18 | | |
|---|---|---|
| comparative | Example 19 | Example 20 |
| 72 | 86 | 120 |

An improvement in the resistance to solvents is recorded for the compositions of the invention.

Impact strength: all the varnishes obtained from the compositions of the above examples pass the test.

In conclusion, it is thus seen, from the tests given above, that the compositions of the invention markedly improve the drying time and it is found that the other properties of the film are not detrimentally affected and may even be improved.

### EXAMPLES 21 TO 23

These examples relate to the use of a comparative curing composition and curing compositions according to the invention in an application of 2K varnish type in the solvent phase for general industrial use.

The characteristics of the curing compositions, which are composed of a part A based on a polyol and of a part B based on a comparative compound having isocyanate functionality or compounds having isocyanate functionality according to the invention, are given in table 8 below.

In the case of example 21, the comparative compound having isocyanate functionality is Tolonate XFD, which is mentioned above. Examples 22 and 23 comprise, as compounds having isocyanate functionality, the compounds of examples 2 and 3 above respectively.

TABLE 8

| | Example 21 | | |
|---|---|---|---|
| | comparative | Example 22 | Example 23 |
| Part A | | | |
| Synocure 852 BA 80 | 78.00 | 78.00 | 76.00 |
| DBDL, Fluka (1% in | 1 | 1 | 1 |
| butyl acetate) | | | |
| Butyl acetate | 41 | 43.4 | 46.7 |
| Part B | | | |
| Tolonate XFD | 41.20 | | |
| Compound of example 2 | | 36.80 | |
| Compound of example 3 | | | 46.50 |

Crosslinking is carried out at 23° C. with 55% relative humidity. The viscosity with the DIN 4 cup is adjusted to 23 s.

The properties of the varnishes obtained, measured according to different tests, are given below.

Pot life: the results of this test are given in the following table 9.

TABLE 9

| | Example 21 | | |
|---|---|---|---|
| | comparative | Example 22 | Example 23 |
| Pot life (min) | 35 | 60 | 70 |

A marked improvement in the cases of the compositions according to the invention is recorded.

Drying time: the results of this test are given in the following table 10.

TABLE 10

| | Example 21 | | |
|---|---|---|---|
| | comparative | Example 22 | Example 23 |
| T2 (min) | 450 | 385 | 330 |
| T3 (min) | 545 | 530 | 435 |

A significant reduction in the drying times T2 and T3 is recorded for the compositions according to the invention.

Gloss: the results of this test (carried out here after drying for 3 days) are given in the following table.

TABLE 11

| Example 21 | | |
|---|---|---|
| comparative | Example 22 | Example 23 |
| 97 | 96 | 98 |

All the compositions exhibit a satisfactory appearance.

Persoz hardness: the results of this test are given in the following table 12.

TABLE 12

| | Example 21 | | |
|---|---|---|---|
| Time | comparative | Example 22 | Example 23 |
| 1 day | 150 | 167 | 205 |
| 3 days | 197 | 215 | 245 |
| 7 days | 252 | 256 | 303 |

The values obtained for the compositions of the invention are greater throughout the formation of the film, very particularly in the case of example 23.

Here again, in conclusion, it is recorded that the compositions of the invention improve the drying time without detrimentally affecting the other properties.

### EXAMPLES 24 AND 25

These examples relate to the use of a comparative curing composition and a curing composition according to the invention in an application of 2K varnish type in the solvent phase for general industrial use.

The characteristics of the curing compositions, which are composed of a part A based on a polyol and of a part B based on a comparative compound having isocyanate functionality or a compound having isocyanate functionality according to the invention, are given in table 13 below.

In the case of example 24, the compound having isocyanate functionality used is that of example 3. The compound having isocyanate functionality used in comparative example 25 is Tolonate XFD, which has been mentioned above.

The crosslinking and drying conditions are modified. Crosslinking is carried out with 10′ of desolvation at ambient temperature (flashoff) and 30′ at 60° C. No catalyst is used.

TABLE 13

| | | Example 25 |
|---|---|---|
| | Example 24 | comparative |
| Part A | | |
| Synocure 852 BA 80 | 76.00 | 78.00 |
| DBDL, Fluka (1% in butyl | 2 | 2 |
| acetate) | | |
| Butyl acetate | 43.3 | 41.4 |
| Part B | | |
| Compound of Example 3 | 46.50 | |
| Tolonate XFD | | 40.50 |

The results of the different tests carried out on the varnishes obtained are given in the summarizing table 14 below.

TABLE 14

| | | | | | Methyl |
|---|---|---|---|---|---|
| | Persoz | Persoz | | | ethyl |
| | hardness | hardness | | AFNOR | ketone |
| | 1 day | 7 days | Gloss | impact | test |
| Example 25 | 48 | 301 | 96 | 100 | 90 |
| comparative | | | | | |
| Example 24 | 65 | 313 | 97 | 100 | 182 |

### EXAMPLES 26 AND 27

These examples relate to the use of a comparative curing composition and a curing composition according to the invention in an application of 2K varnish type in the solvent phase for general industrial use.

The compound having isocyanate functionality used in comparative example 26 is a mixture based on HDI and on IPDI in the trimeric form in a respective ratio by weight of 60/40. In the case of example 27, the compound having isocyanate functionality used is that of example 3.

Crosslinking is carried out at 23° C. with 55% relative humidity.

The characteristics of these various curing compositions are combined in the following table 15.

TABLE 15

| | Example 26 | |
|---|---|---|
| | comparative | Example 27 |
| NCO compound having isocyanate | 17.27% | 14.9% |
| functionality (as is) | | |
| Solids extract compound having | 85.4% | 100% |
| isocyanate functionality | | |

The results of the various tests carried out on the varnishes obtained are given below.

### Gloss:

There is no difference between the compositions evaluated and all exhibit a satisfactory appearance (20° gloss >97).

### Persoz Hardness:

TABLE 16

| | Example 26 | |
|---|---|---|
| | comparative | Example 27 |
| 1 day | 110 | 100 |
| 3 days | 194 | 181 |

### Impact Strength:

TABLE 17

| | Example 26 | |
|---|---|---|
| | comparative | Example 27 |
| AFNOR D7 | 40 cm | 100 cm |
| ASTM D7 | 35 cm | 80 cm |

The difference is very marked for the mixture of the comparative example, which is very brittle in comparison with the mixture according to the example of the invention.

### EXAMPLES 28 TO 30

These examples relate to the use of comparative curing compositions and a curing composition according to the invention in an application of 2K varnish type in the solvent phase for general industrial use. The characteristics of the curing compositions, which are composed of a part A based on a polyol and of a part B based on comparative compounds having isocyanate functionality or a compound having isocyanate functionality according to the invention, are given in table 18 below.

These examples demonstrate the importance of the preliminary reaction of the polyisocyanate with the compound having a labile hydrogen in comparison with the simple physical addition of the latter in part A.

In the case of example 28, the compound having isocyanate functionality used is that of example 3. In the case of example 29, the comparative compound having isocyanate functionality is Tolonate XFD 90 B, which is an aliphatic isocyanate of high functionality having good drying properties. In the case of example 30, the comparative compound is Tolonate HDT LV2 and, in this case, the part A has added to it an amount of HBPA equal to the amount used to synthesize the compound of example 3.

Crosslinking is carried out at 23° C. with 55% relative humidity.

The characteristics of these various curing compositions are combined in the following table 18.

TABLE 18

| | | Example 29 | Example 30 |
|---|---|---|---|
| | Example 28 | comparative | comparative |
| Part A | | | |
| Synocure 852BA80 | 74.22 | 70.91 | 68.78 |
| HBPA | | | 2.99 |
| DBDL, Fluka (1% in BuOAc) | 2.00 | 1.82 | 1.77 |
| Butyl acetate | 23.79 | 27.27 | 26.46 |
| Part B | | | |
| Compound of example 3 | 43.00 | | |
| Tolonate XFD | | 36.00 | |
| Tolonate HDT LV2 | | | 31.90 |
| Butyl acetate | 5.00 | 5.00 | 5.00 |

The results of the various tests carried out on the varnishes obtained are given below.

### Pot Life:

TABLE 19

| | | Example 29 | Example 30 |
|---|---|---|---|
| | Example 28 | comparative | comparative |
| Pot life (min) | 55 | 40 | 40 |

The pot life obtained for the mixture of example 28 is better than in the case of the comparative examples 29 and 30.

### Drying Time:

TABLE 20

| | | Example 29 | Example 30 |
|---|---|---|---|
| | Example 28 | comparative | comparative |
| T1 (min) | 60 | 75 | 280 |

The addition of the HBPA with part A is reflected by a very high dust-dry drying time compared with the compound of the invention. The preliminary reaction between the polyisocyanate and the HBPA is essential in order to obtain improved drying properties.

The examples which follow illustrate compounds according to the invention and their use in compositions of the curing agent type in the aqueous phase.

In these examples, use is made, as starting material, of the products Rhodafac DV 6175 and Rhodafac DV 6176, which are mixtures of surfactants, sold by Rhodia, composed of a mixture of phosphate mono- and diester of a polyethylene glycol fatty alcohol monoether and of phosphoric acid. The fatty alcohol is a branched chain composed of 13 carbon atoms on average and the number of ethylene oxide units is centered around 6 approximately. These two surfactants differ in the ratio of the monoester, diester and phosphoric acid compounds.

For the compositions in the aqueous phase, the size of the particles of the hydrophilic polyisocyanates after dispersion in the water is measured in the following way: 5 g of the hydrophilic polyisocyanate formulation are added to 45 g of distilled water at ambient temperature. The reaction medium is stirred at 400 revolutions/minute for 5 minutes using a stirring module of 4-bladed propeller type.

The particle size curve is measured using a laser diffraction particle sizer of Malvern Mastersizer 2000 type.

### EXAMPLE 31

This example relates to the preparation of a compound X (phase 1); the preparation of a compound according to the invention from the compound X formed above (phase 2); the preparation of a composition or formulation of curing agent type according to the invention in the aqueous phase (phase 3).

### Phase 1

The compound X prepared here is a diol ester which is the product of the reaction of a compound having an epoxy functional group with a phosphate.

50.76 g of dibutyl phosphate (i.e. 0.242 mol/CAS RN 107-66-4) are introduced into a jacketed three-necked reactor equipped with a mechanical stirrer and with dropping funnels. 29.94 g of 3,4-epoxycyclohexylmethyl 3,4-epoxycyclohexanecarboxylate (i.e. 0.119 mol/CAS RN 2386-87-0) are added over 20 minutes at ambient temperature with stirring and under a nitrogen atmosphere. The reaction is exothermic. The temperature of the reaction medium is brought to 100° C. The reaction kinetics are monitored by infrared (IR) analysis carried out on a withdrawn sample of the reaction medium.

The disappearance of the epoxy functional groups is monitored at 788.76 cm⁻¹. The appearance of the hydroxyl functional groups is observed at 3392.96 cm⁻¹ and that of the phosphate ester functional groups is observed at 1253.59 cm⁻¹. The carboxylic ester functional group of the starting material is displayed at 1732.88 cm⁻¹. The alkyl band situated at 2955 cm⁻¹ is used as reference. Kinetic monitoring is measured on the ratio of the frequencies 788.76 cm⁻¹/2955 cm⁻¹.

The reaction is terminated as soon as the IR analysis shows that the epoxy functional group is more than 95% open and that the expected diol ester is indeed obtained.

As the epoxy functional group can be opened by attack on the 3 carbon or the 4 carbon, the final diol ester product is a mixture of structures, one of which is represented below.

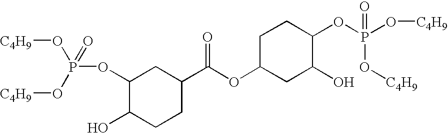

### Phase 2

13.82 g of the diol ester obtained on conclusion of phase 1 (0.082 mol of hydroxyl functional groups) and 302 g of isocyanurate polyisocyanate Tolonate HDT LV2, the NCO assay of which is 0.543 mol of NCO per 100 g and the viscosity of which at 25° C. is 600 mPa·s, are introduced into a jacketed three-necked reactor equipped with a mechanical stirrer and with dropping funnels.

The diol ester/polyisocyanate ratio by weight is 4.6%.

The temperature of the reaction medium is then raised to 100° C. and the reaction medium is left stirring at this temperature for 5H 30. After cooling, the product is collected in a receiving flask. The weight recovered is 315 g. The viscosity is 1850 mPa·s at 25° C.

As Tolonate HDT LV2 is a formulation of polyisocyanates comprising several structures and as the product resulting from phase 1 is itself also a mixture of structures, the final product obtained is a formulation of polyisocyanate urethane esters, one of the structures of which is represented below.

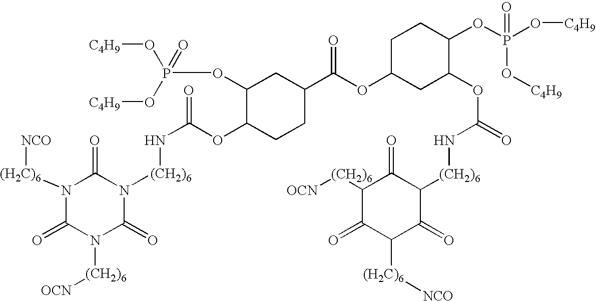

### Phase 3

90 g of the product resulting from phase 2, 1.92 g of N,N-dimethylcyclohexylamine, 4.04 g of Rhodafac DV 6175 surfactant and 4.04 g of Rhodafac DV 6176 surfactant are successively introduced into a reactor. The mixture is stirred at 40° C. for 2 hours and then cooled to ambient temperature.

The NCO assay of the final formulation of curing agent type based on hydrophilic polyisocyanate thus obtained is 0.466 mol of NCO per 100 g, i.e. 19.57% by weight.

The particle size, measured after dispersion in water, is centered on 0.1 micron.

### EXAMPLE 32

This example also relates to the preparation of a compound X (phase 1); to the preparation of a compound according to the invention from the compound X formed above (phase 2); to the preparation of a composition of curing agent type according to the invention in the aqueous phase (phase 3).

### Phase 1

The compound X prepared here is a dial ester, which is the product of the reaction of a compound having an epoxy functional group with a carboxylic acid.

20 g of 3,4-epoxycyclohexylmethyl 3,4-epoxycyclohexanecarboxylate (i.e., 0.079 mol/CAS RN 2386-87-0) are introduced into a jacketed three-necked reactor equipped with a mechanical stirrer and with dropping funnels. 12.09 g of propionic acid (0.165 mol) are added over 20 minutes at ambient temperature with stirring and under a nitrogen atmosphere. The reaction is exothermic. The temperature of the reaction medium is brought to 100° C.

After 3 hours at 100° C., it is observed, by infrared analysis, that the epoxy compound is more than 92% open and that the expected dial is indeed obtained. The reaction medium is left stirring at 100° C. for a further 4 hours, is then allowed to cool to ambient temperature and is then transferred into a flask.

The product is monitored by infrared:
disappearance of the epoxy functional groups (788.76 cm⁻¹)
appearance of the hydroxyl functional groups (3392.96 cm⁻¹)
confirmation of the carboxylic ester functional group (1732.88 cm⁻¹).

As the epoxy functional group can be opened by attack on the 3 carbon or the 4 carbon, the final diol ester product is a mixture of structures, one of which is represented below.

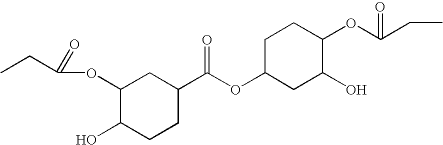

### Phase 2

10.9 g (0.027 mol) of the dial ester obtained on conclusion of phase 1, with a molecular weight of 400.47, and 303.8 g of isocyanurate polyisocyanate Tolonate HDT LV2, with a viscosity at 25° C. of 600 mPa·s and with an NCO assay of 0.543 mol of NCO per 100 g, are introduced into a jacketed three-necked reactor equipped with a mechanical stirrer and with dropping funnels.

The diol ester/polyisocyanate ratio by weight is 4%. 15 microliters of dibutyltin dilaurate are added. The temperature of the reaction medium is then brought to 110° C. and the reaction medium is left stirring at this temperature for 8H. The NCO assay changes from 0.525 mol per 100 g to 0.492 mol of NCO per 100 g after reacting for 8H. The consumption of the NCO functional groups represents 6.3% at the end of the reaction.

After cooling, the product is collected in a flask. The weight recovered is 314 g.

The NCO assay of the final formulation of polyisocyanates is 0.492 mol of NCO per 100 g. The viscosity is 5187 mPa·s at 25° C.

As Tolonate HDT LV2 is a formulation of polyisocyanates comprising several structures and as the product resulting from phase 1 is itself also a mixture of structures, the final product obtained is a formulation of polyisocyanate urethane esters, one of the structures of which is represented below.

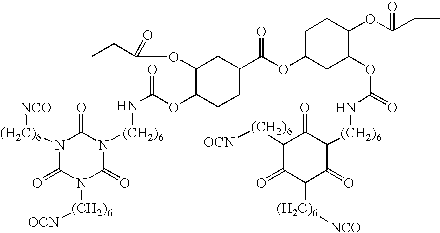

### Phase 3

90 g of the product resulting from phase 2, 1.92 g of N,N-dimethylcyclohexylamine, 4.04 g of Rhodafac DV 6175 surfactant and 4.04 g of Rhodafac DV 6176 surfactant are successively introduced into a reactor. The mixture is stirred at 40° C. for 2 hours and then cooled to ambient temperature.

The NCO assay of the final formulation of curing agent type based on hydrophilic polyisocyanate is 0.418 mol of NCO per 100 g, i.e. 17.56% by weight. The particle size, measured after dispersion in water, is centered on 0.1 micron.

### COMPARATIVE EXAMPLE 33

This example describes the preparation of a formulation from an unmodified Tolonate HDT LV2, by way of comparison with the formulations obtained on completion of phase 3 of examples 31 and 32.

90 g of Tolonate HDT LV2, 1.92 g of N,N-dimethylcyclohexylamine, 4.04 g of Rhodafac DV 6175 surfactant, 4.04 g of Rhodafac DV 6176 surfactant and 30 g of methoxypropyl acetate are introduced into a reactor. The mixture is stirred at 40° C. for 2 hours and is then cooled to ambient temperature.

The NCO assay of the final formulation obtained is 0.36 mol per 100 g.

### EXAMPLE 34

This example illustrates the use of a comparative curing agent composition and curing agent compositions according to the invention in applications of varnish type.

A varnish formulation is produced by using, as starting materials, a dispersion of an acrylic polyol, the level of hydroxyl functional groups of which is 4.8%, and the formulations obtained on conclusion of phase 3 of examples 31 and 32 and comparative example 33. The NCO/OH molar ratio used is 1.2.

50 g of polyol, 14.54 g of formulation of example 31 obtained on conclusion of phase 3, 3.91 g of water and 0.012 g of dibutyltin dilaurate (DBTL) are successively added to a reactor. The mixture is stirred for 2 minutes with moderate stirring. The formulation obtained is homogeneous and no aggregate is visible. Foam formation does not occur. The emulsion is left standing for 15 minutes before any application to the support.

The procedure is the same for the formulation of hydrophilic polyisocyanate curing agent of example 32 obtained on conclusion of phase 3.50 g of polyol, 16.21 g of said formulation, 4.01 g of water and 0.011 g of dibutyltin dilaurate (DBTL) are successively added to a reactor. The mixture is stirred for 2 minutes with moderate stirring. The formulation obtained is homogeneous and no aggregate is visible.

Foam formation does not occur. The emulsion is left standing for 15 minutes before any application to the support.

The comparative formulation is prepared in the same way by adding, to a reactor, 50 g of polyol, 18.77 g of formulation of comparative example 33, 4.17 g of water and 0.014 g of DBTL. The emulsion is left standing for 15 minutes before any application to the support.

The formulations are subsequently applied to glass plates. A scraper with a thickness of 150 μm and a film drawer are used to spread the film. The scraper is positioned on the plate, is filled with the emulsion and is pushed by the film drawer at a speed of 24 mm/s.

After evaporating at ambient temperature for 15 minutes, the plates are placed in an oven at 60° C. for 30 minutes. On completion of the curing, the properties of the films are then measured after drying at ambient temperature for 1, 3 and 24 hours.

The results are presented in table 21 below.

TABLE 21

| | Film | | | | |
|---|---|---|---|---|---|
| Curing | thickness | Hardness | Hardness | Hardness | |
| agent | in microns | 1 H | 3 H | 24 H | Gloss |
| Ex. 31 | 33 | 68 | 108 | 315 | 92 |
| Ex. 32 | 32 | 82 | 130 | 315 | 90 |
| Ex. 33 | 29 | 27 | 54 | 305 | 92 |
| comparative | | | | | |

The formulations of hydrophilic polyisocyanate curing agents of the invention obtained from Tolonate HDT LV2 modified by reaction with cycloaliphatic groups exhibit better film properties than the same unmodified polyisocyanate. It is very particularly observed that the initial hardness (hardness at 1 and 3 hours) is much higher.

1.-21. (canceled)

22. A compound bearing at least one isocyanate functional group substituent having a mean functionality of greater than 2, comprising the product of reaction between a (poly)isocyanate having a mean functionality of greater than 2 and at least one compound X which comprises:
either at least one functional group B(H)ₙ in which:
n is a number equal to 1 or 2,
H is a labile hydrogen atom and
B is O, S, N (wherein N is a primary or secondary nitrogen atom), —C(═O)—O, —C(═O)—N, or a group: 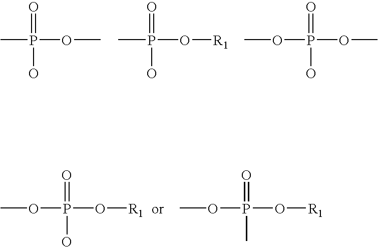
R₁ is an alkyl or aralkyl radical which is optionally branched or an alkyl radical interrupted by a heteroatom;
or at least one functional group B′(H)_{n′} in which:
n′ is a number equal to 1, 2 or 3,
H is a labile hydrogen atom and
B′ is —SiR₂R₃R₄, wherein R₂, R₃ and R₄ are each oxygen, an alkyl radical substituted by a functional group which reacts with the (poly)isocyanate, or an aralkyl, aryl, —O-alkyl or —O-aralkyl radical, the number of R₂, R₃ and R₄ radicals being such that n′ is indeed as above defined;
with the proviso that the compound X can be a cycloaliphatic, aromatic or heterocyclic compound or a particle of a crosslinked polymer;
and with the further provisos:
that, when B is a secondary nitrogen atom and when the compound X is a cycloaliphatic compound, the compound X contains at least two ring members;
and that said reaction is carried out with a compound X/[compound X+(poly)isocyanate] ratio by weight of at most 50%.

23. The compound as defined by claim 22, having a functionality of at least 2.5.

24. The compound as defined by claim 22, wherein the compound X has a rigid structure.

25. The compound as defined by claim 22, wherein the at least one compound X comprises at least one functional group B(H)ₙ or B′(H)_{n′} bonded directly to a carbon atom of a ring member thereof.

26. The compound as defined by claim 22, wherein the at least one compound X comprises only a single ring member and the at least one functional group B(H)ₙ or B′(H)_{n′} is bonded directly to a carbon atom of this ring member.

27. The compound as defined by claim 22, wherein the at least one compound X comprises at least one functional group B(H)ₙ which is an OH functional group.

28. The compound as defined by claim 27, wherein the at least one compound X is selected from the group consisting of:
a bisphenol or hydrogenated or polyphenolic derivative thereof having an ether bridge and a hydrogenated derivative of the latter;
a cyclopentadiene, dicyclopentadiene and tricyclopentadiene derivative thereof;
a terpene derivative;
a cycloalkane having an OH functional group substituent.

29. The compound as defined by claim 27, wherein the at least one compound X is the product of the reaction of a carboxylic acid with a compound having a blocked hydroxyl functional group substituent.

30. The compound as defined by claim 27, wherein the at least one compound X is the product of the reaction of a carboxylic acid with a compound having an epoxy functional group substituent.

31. The compound as defined by claim 27, wherein the at least one compound X is the product of the reaction of a compound comprising at least one epoxy functional group substituent with a phosphate of formula (O)P(OR₆)(OR₇)(OR₈) in which R₆, R₇ and R₈, which may be identical or different, are each hydrogen, a linear or branched alkyl radical, a cycloaliphatic radical, an aromatic radical, an aralkyl radical or a polyoxyalkylene radical, the number of linear or branched oxyalkylene units of which ranges from 1 to 25 and the number of carbons of the alkylene radical of which ranges from 2 to 6, with the proviso that this polyoxyalkylene radical may be substituted on the end functional group thereof by a linear or branched alkyl radical or by an aralkyl radical which is optionally branched; and with the further proviso that at least one of R₆, R₇ and R₈ is a hydrogen atom.

32. The compound as defined by claim 27, wherein the at least one compound X is the product of the reaction of a compound having an epoxy functional group substituent with a polyaminoether of formula (3) 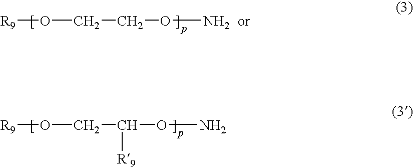
or also of formula (4) R₉—[—O—CH₂—CH₂—O—]_{q}—CH(CH₃)—CH₂—NH₂, in which R₉ is a hydrogen atom, an alkyl radical or a CH₂CH₂NH₂ or CH₂CH(CH₃)NH₂ radical, R′₉ is an alkyl radical, and p and q are integers ranging from 2 to 10.

33. The compound as defined by claim 22, wherein the at least one compound X is reacted with a (poly)isocyanate having at least one isocyanurate ring member or one biuret structural unit or one allophanate functional group or also one acylurea functional group and which is prepared by homo- or heterocondensation of monomers selected from the group consisting of aliphatic, cycloaliphatic, arylaliphatic, aromatic and heterocyclic isocyanate monomers.

34. A process for the preparation of a compound as defined by claim 22, comprising reacting a (poly)isocyanate with a compound X in a compound X/[compound X+(poly)isocyanate] ratio by weight of at most 40%.

35. The process as defined by claim 34, comprising reacting the (poly)isocyanate with the compound X in an amount such that the B(H)ₙ/NCO molar ratio ranges from 1 to 50%.

36. A curing agent composition comprising at least one compound as defined by claim 22.

37. The curing agent composition as defined by claim 36, comprising an unreactive hydrophilic additive.

38. The curing agent composition as defined by claim 36, comprising a hydrophilic additive grafted to the compound having isocyanate functional group substituents.

39. The curing agent composition as defined by claim 36, further comprising a polyisocyanate.

40. A process for depositing a coating onto a substrate, comprising reacting a composition as defined by claim 36, with a compound substituted by at least one functional group having a mobile hydrogen atom selected from among hydroxyl, primary amine and secondary amine functional groups and the SH functional group or with a compound comprising precursor functional groups capable of releasing hydroxyl functional groups, and then applying the mixture obtained onto the substrate.

41. The process as defined by claim 40, wherein the compound substituted by at least one functional group having a mobile hydrogen comprises a polyol selected from among acrylic, polyester or polyurethane polymers or blends of these polymers.

42. A drying accelerator comprising a compound as defined by claim 22, and wherein a composition containing said compound comprises a mixture with a compound substituted with at least one functional group having a mobile hydrogen atom selected from among hydroxyl, primary amine and secondary amine functional groups and the SH functional group.
